(19) United States
(12) Patent Application Publication
(10) Pub. No.: US 2026/0137748 A1
(43) Pub. Date: May 21, 2026

(54) APPLICATION OF WITHANIA SOMNIFERA IN COMBINATION WITH MELATONIN

(71) Applicant: NANJING HAIGUANG INSTITUTE OF APPLIED CHEMISTRY CO. LTD, Nanjing (CN)

(72) Inventors: Jingfei Ma, Nanjing (CN); Hongyu Chen, Nanjing (CN); Tingting Song, Nanjing (CN)

(21) Appl. No.: 19/222,351

(22) Filed: May 29, 2025

(30) Foreign Application Priority Data
Nov. 21, 2024 (CN) 202411677593.5

(51) Int. Cl. A61K 36/81; A61K 31/4045; A61P 25/20; A61P 25/22

(52) U.S. Cl. CPC A61K 36/81; A61K 31/4045; A61P 25/20; A61P 25/22

(57) ABSTRACT

The application relates to a combined use of Withania somnifera and Melatonin. In the application, after using Withania somnifera and Melatonin in combination, they have significantly synergistic effects in improving sleep and/or improving emotion. Especially, the effects of improving sleep and/or improving emotion achieved by the way of administrating Withania somnifera in the morning in combination with administrating Melatonin in the evening are better than the effects achieved by simultaneously administrating them in the evening and by administrating each of them alone.

### CROSS-REFERENCE TO RELATED APPLICATIONS

The present application is based on and claims the benefit of priority from Chinese Application No. 202411677593.5 filed on Nov. 21, 2024, the disclosures of which are incorporated herein by reference in its entirety.

### TECHNICAL FIELD

The present application relates to the combined use of Withania somnifera and Melatonin, particularly to the combined use of Withania somnifera and Melatonin for improving sleep and/or improving emotion.

### BACKGROUND ART

In modern living or working environments of high-development and high-stress, the number of patients suffered with insomnia and anxiety disorder exhibits a rising tendency, and these diseases seriously affect daily work and lives and become non-negligible diseases.

### SUMMARY

Melatonin (MT), with the chemical name N-acetyl-5-methoxytryptamine, has the physiological effect of participating in the regulation of circadian rhythm and sleep in human body. In recent years, the Melatonin has been widely used in the treatment of various sleep disorders, and a large number of clinical research have been made. Withania somnifera (WS), also called as Indian ginseng and Ashwagandha, is a traditional medicine in Indian Ayurveda medical system and it is often used for anti-anxiety, sleep aid, anti-aging, etc. The British Pharmacopoeia has recorded that Withania somnifera root may be used as a Traditional Herbal Medicinal Product (THMP); as a dietary supplement, an extract of Withania somnifera root is generally used.

Through long-term research, the inventors have unexpectedly found that the combined use of Withania somnifera and Melatonin has the effect of improving sleep and/or improving emotion, and both of them have significant synergistic effects in improving sleep and/or improving emotion. Especially, the effects of improving sleep and/or improving emotion achieved by administrating Withania somnifera in the morning in combination with administrating Melatonin in the evening are better than the effects achieved by simultaneously administrating them in the evening or by administrating each of them alone. The above research result provides a theoretical basis for clinical applications and has excellent practical application values.

One aspect of the present application provides a use of a combination of Withania somnifera and Melatonin in the manufacture of a medicament or a dietary supplement, wherein the Withania Somnifera is administered in the morning and the Melatonin is administered in the evening.

The combination has the function of improving sleep and/or improving emotion. The medicament or the dietary supplement has the function of improving sleep and/or improving emotion.

A second aspect of the present application provides a method for administering Withania Somnifera and Melatonin to a subject in need thereof, wherein the Withania somnifera is administrated in the morning and the Melatonin is administrated in the evening.

In certain embodiments, the Withania somnifera is a medicament or a dietary supplement. In certain embodiments, the Melatonin is a medicament or a dietary supplement.

In certain embodiments, the method is used for improving sleep and/or emotion of the administrated subject.

A third aspect of the present application provides a combined package of Withania Somnifera and Melatonin, wherein the Withania somnifera is a medicament or dietary supplement and is administered in the morning; the Melatonin is a medicament or a dietary supplement and is administered in the evening.

In certain embodiments, the combined package is used for improving sleep and/or emotion of the administrated subject.

A fourth aspect of the present application provides a method for improving sleep and/or improving emotion, comprising administering an effective amount of Withania somnifera and Melatonin to a subject in need thereof, wherein the Withania somnifera is administered in the morning, and the Melatonin is administered in the evening.

In certain embodiments, the Withania somnifera is administered in the form of a medicament or a dietary supplement. In certain embodiments, the Melatonin is administered in the form of a medicament or a dietary supplement.

In certain embodiments, the improving sleep includes, but is not limited to, shortening sleep latency, and increasing sleep time.

In certain embodiments, the improving emotion includes, but is not limited to, alleviating anxiety, relieving tension, reducing stress, and enhancing mood pleasure.

In certain embodiments, the Withania somnifera of the present application is an extract of Withania somnifera root.

In certain embodiments, the Withania somnifera of the present application is an extract of Withania somnifera root; the extract of the Withania somnifera root is administrated in an amount of 100 mg to 1500 mg per day.

In certain embodiments, the Withania somnifera of the present application is an extract of Withania somnifera root; the extract of the Withania somnifera root is administrated in an amount of 120 mg to 1250 mg per day. The journal article (J Funct Morphol Kinesiol, 2021, 6, 20) has disclosed that taking the Withania somnifera as dietary supplement in an amount of 120 mg to 1250 mg per day can enhance physical functions. The British Pharmacopoeia (2009) records that “Withania somnifera Root for use in THMP” (THMP is the abbreviation for Traditional Herbal Medicinal Product), and it specifies that the Withania somnifera root contains not less than 0.01% of withaferin (C₂₈H₃₈O₆) and not less than 0.01% of withanolide (C₂₉H₄₂O₇). The extract of Withania somnifera root is prepared from Withania somnifera root, wherein the content of active ingredients is increased by concentration. Illustratively, KSM-66® is a commercialized extract of Withania somnifera root, which may be used as a standalone product or as a supplemental ingredient in various formulations of herbal and dietary supplements, with a withanolide content of about 5%. In certain embodiments, the extract of Withania somnifera root of the present application is prepared from Withania somnifera roots, and the withanolide content in the extract of Withania somnifera root is 1.5% to 10% (preferably 2.5% to 6%).

In certain embodiments, the Melatonin of the present application is administrated in an amount of 0.3 mg to 15 mg per day.

In certain embodiments, the Melatonin of the present application is administered in an amount of 1 mg to 10 mg per day. The SmPC/label of the medicament approved by European Medicines Agency (EMA) indicate that the Melatonin will not cause any significant adverse effects at a daily dose of up to 300 mg.

In the present application, unless otherwise specified, the administrated subject refers to human.

The Withania somnifera or Melatonin of the present application may be present in a medicament or a dietary supplement in the form of a unit dose, to facilitate the administration thereof.

As used in the present application, the term “medicament” refers to any substance that provides therapeutic, preventive and/or beneficial effects.

As used in the present application, the term “dietary supplement” refers to an edible product capable of providing beneficial effects to consumers, or to a dietary supplement as defined in the Dietary Supplement Health and Education Act (1994) (DSHEA). For Example, DSHEA defines a dietary supplement as a product (other than tobacco) intended to supplement the diet which contains one or more of the dietary ingredients including vitamins, minerals, herbs or other botanicals, amino acids, or other known dietary substances, with the form of pills, capsules, tablets, or liquids.

In the present application, the medicament or the dietary supplement comprises Withania Somnifera or Melatonin as the active ingredient, and a pharmaceutically acceptable carrier and/or excipient. The medicament or the dietary supplement may be formulated into any dosage forms known in the medical field, such as tablets, pills, suspensions, emulsions, solutions, gels, capsules, powders, granules, elixirs, lozenges, suppositories, injections (including injection liquids, sterile powders for injection and concentrated solutions for injection), inhalants, sprays and the like. Preferred dosage forms depend on intended administration route and therapeutic purpose.

In the present application, the medicament may be administered by any suitable route known in the art, including, but being not limited to, oral, buccal, sublingual, ophthalmic, topical, parenteral, rectal, intrathecal, intracisternal, inguinal, intravesical, topical (e.g., powder, ointment, or drops), or nasal routes. However, for many therapeutic uses, the preferred administration route/way is an oral, buccal or sublingual administration. Those skilled in the art should understand that the administration route and/or way will vary depending on intended purposes. In some preferred embodiments, the medicament is administered orally.

In the present application, the dietary supplement may be administered by any suitable route known in the art, including, but being not limited to, oral, buccal, and sublingual. In some preferred embodiments, the dietary supplement is administered orally.

As used in the present application, the term “pharmaceutically acceptable carrier and/or excipient” refers to a carrier and/or excipient that is pharmacologically and/or physiologically compatible with subjects and active ingredients, which is well known in the art (see, e.g., Remington's Pharmaceutical Sciences, Edited by Gennaro AR, 19th ed. Pennsylvania: Mack Publishing Company, 1995) and includes, but is not limited to, pH regulators, surfactants, adjuvants, ionic strength enhancers, diluents, osmotic pressure maintaining agents, absorption-delaying agents, and preservatives. For example, the pH regulators include, but are not limited to, phosphate buffers. The surfactants include, but are not limited to, cationic, anionic or nonionic surfactants, e.g., Tween-80. The ionic strength enhancers include, but are not limited to, sodium chloride. The preservatives include, but are not limited to, various antibacterial agents and antifungal agents, such as parabens, chlorobutanol, phenol, sorbic acid and the like. The osmotic pressure maintaining agents include, but are not limited to, sugars, NaCl, and analogues thereof. The absorption-delaying agents include, but are not limited to, monostearate salts and gelatin. The diluents include, but are not limited to, water, aqueous buffers (e.g., buffered saline), alcohols and polyols (e.g., glycerol) and the like. The stabilizers have the meanings as commonly understood by those skilled in the art, and they can stabilize the desired activities of active ingredients in a medicament, including, but being not limited to, sodium glutamate, gelatin, SPGA, sugars (such as sorbitol, mannitol, starch, sucrose, lactose, dextran, or glucose), amino acids (such as glutamic acid, or glycine), proteins (such as dried whey, albumin, or casein) or degradation products thereof (such as lactalbumin hydrolysate) and the like.

The term “morning” in the present application should be understood in terms of daily life. Illustratively but not exclusively, the “morning” may be understood as a period from sunrise to five hours after sunrise.

The term “evening” in the present application should be understood in terms of daily life. Illustratively but not exclusively, the “evening” may be understood as a period from sunset to eight hours after sunset.

As used in the present application, the term “anxiety” refers to a specific psychological state involving extreme fear or worry. Its symptoms include excessive worry, nervousness, fear of future events and the like.

As used in the present application, the term “insomnia” refers to a common health problem that affects sleep quality and quantity of an individual. The clinical symptoms of the insomnia are mainly manifested by difficulty in falling asleep, frequent waking during the night, early waking and difficulty in falling asleep again, feeling tired and unable to concentrate after waking up and the like.

The manifestations of the “improving sleep” in the present application include, but not are limited to, shortening sleep latency and increasing sleep time.

The manifestations of the “improving emotion” in the present application include, but are not limited to, alleviating anxiety, relieving tension, reducing stress, and enhancing mood pleasure; it should be illustrated that these manifestations are not completely in a parallel relationship, but overlap each other to some extent.

As used in the present application, the term “combination” includes both a combined package of said substances for the combined administration, and a combined therapy/administration method of said substances.

As used in the present application, the term “effective amount” refers to an amount sufficient to improve sleep or improve emotion. In the present application, the effective amount of Withania Somnifera or Melatonin may vary depending on the following factors: emotional and sleep status of administrated subjects, overall conditions of administrated subjects, general conditions such as age, body weight and gender, administration route of medicaments or dietary supplements, and other concurrently-administrated treatments and the like.

In general, in comparison to the prior art, the above technical solutions conceived by the present application can achieve the following beneficial effects:

The present application, through experiments, has proved that the combined use of the administration of Withania somnifera in the morning and the administration of Melatonin in the evening can significantly improve sleep and/or improve emotion, and the effect of improving sleep and/or improve emotion achieved by the way of administrating Withania somnifera in the morning in combination with administrating Melatonin in the evening are better than the effects achieved by simultaneously administrating them in the evening and by administrating each of them alone. The research result of the present application can have excellent practical application values in improving sleep and/or improving emotion.

### BRIEF DESCRIPTION OF THE DRAWINGS

The accompanying drawings illustrated here are intended to provide further understandings to the present application, and they constitute a part of the present application. The illustrative examples of the present application and the illustrations thereto are used for explaining the present application, and they do not constitute improper limitations for the present application.

FIG. 1 shows the effects of the combined use of Withania somnifera (WS) and Melatonin (MT) on the mouse body weight (1^{st} day to 14^{th} day);

FIG. 2 shows the effects of the combined use of Withania somnifera (WS) and Melatonin (MT) on the total journey (A), average speed (B) and rest time (C) in the spontaneous activity experiment of mice;

FIG. 3 shows the effects of the combined use of Withania somnifera (WS) and Melatonin (MT) on the time spent in open arms (A) and frequency of entering open arms (B) in the elevated plus maze experiment of mice;

FIG. 4 shows the effects of the combined use of Withania somnifera (WS) and Melatonin (MT) on the sleep latency time (A) and sleep time (B) in the pentobarbital-induced sleep experiment of mice;

FIG. 5 shows the effects of the combined use of Withania somnifera (WS) and Melatonin (MT) on the corticosterone (CORT) in mouse serum.

### DETAILED DESCRIPTION OF THE PREFERRED EMBODIMENT

The technical solutions in the examples of the present application will be clearly and completely described below with reference to the drawings and specific examples in the present application. Obviously, the described examples are merely intended to illustrate the present application and are not intended to limit the scope of the present application.

The experimental methods used in the following examples are conventional methods unless otherwise specified.

Materials, reagents and the like used in the following examples are commercially available unless otherwise specified.

SPF-grade male Kunming (KM) mice weighing 22-24 g used in the examples are purchased from Liaoning Changsheng Biotechnology, Co., Ltd. All the mice live under normal light/dark (12 h/12 h) conditions, with an environment temperature of 22±2° C. and a relative humidity of 55%+5%, and in a clean-grade and pathogen-free standard environment. The animals can eat food and drink water freely. The animal experimental protocol complies with the requirements and related regulations of China Animal Ethics Committee and Animal Ethics Committee of Shenyang Pharmaceutical University.

It is necessary to particularly illustrate that due to the inherent biological characteristics and habits of animals themselves, the murine circadian rhythm is contrary to that of humans. Therefore, the administration time points are adjusted accordingly in the experimental studies of the present application.

### Example 1

### 1. Experimental Materials and Methods

### 1.1 Test Substance

Extract of Withania somnifera (WS) root, Batch No.: AskPharma Inc., 0123089; Melatonin (MT), Batch No.: LN12.

### 1.2 Main Apparatus and Reagents

Main apparatus: electronic balance, microplate reader, spontaneous activity monitor, elevated plus maze, and the like.

Main reagents: 0.5% CMC-Na (batch No.: MB1717; purchased from Sigma-Aldrich), diazepam (cat/batch No.: 220506, purchased from Shandong XinYi Pharmaceutical Co., Ltd.), pentobarbital sodium (purchased from LEBO), corticosterone kit (cat/batch No: JL11918; purchased from Jonlnbio).

### 1.3 Experimental Designs and Animal Grouping

SPF-grade male KM mice were selected, weighing 22-24 g, and were purchased from Liaoning Changsheng Biotechnology, Co., Ltd.

After being fed and acclimatized for 2 days, the male mice were randomly divided into 6 groups, with 12 mice per group. The experimental treatment settings were shown in Table 1: (1) blank control group (Control, also abbreviated as Con); (2) Combination group of Melatonin (administrated at 9 AM) and Withania somnifera (administrated at 19 PM) (MT AM+WS PM); (3) Melatonin group (administrated at 9 AM) (MT AM); (4) Withania somnifera group (administrated at 19 PM) (WS PM); (5) Combination group of Melatonin (administrated at 9 AM) and Withania somnifera (administrated at 9 AM) (MT AM+WS AM); (6) positive medicament Diazepam group (administrated at 9 AM) (DZP). Diazepam, being a benzodiazepine medicament, could inhibit the central nervous system and had good effects of anti-anxiety, sedation, hypnosis, anti-convulsion and muscle relaxation.

TABLE 1
Animal grouping and administrating information

| | | | Administration route/ |
|---|---|---|---|
| Groups | Animal number | Dose | frequency |
| Control | 12 | — | Gavage/2 times/day |
| MT AM + WS PM | 12 | MT: 0.6 mg/kg | |
| MT AM | 12 | WS: 0.6 g/kg | |
| WS PM | 12 | | |
| MT AM + WS AM | 12 | | |
| DZP | 12 | 2.0 mg/kg | |

Note:
MT represents Melatonin, WS represents Withania Somnifera.

In the experiment, after normal mice were administrated with the test substance by gavage, the anxiolytic and sleep indicators were detected. All the mice were administrated 2 times per day by gavage (9 AM, 19 PM), and at the time points not for the test substance, each of the mice was administrated with an equal volume of 0.5% CMC-Na; the blank control group was administrated with 0.5% CMC-Na at 9 AM and 19 PM, respectively. On the 14th day, 15th day, and 16th day after the first administration, a spontaneous activity experiment, an elevated maze experiment and a pentobarbital-induced sleep experiment were conducted respectively. The administrations for the experiment continued for 17 days.

### 1.4 Measurement of Body Weights

The body weights were measured daily, recorded and calculated.

### 1.5 Test of Behavioral Indicators

(1) Spontaneous activity experiment: one hour after the administration at 19 PM on the 7^{th} day or 14^{th} day after the first administration, the computer real-time detection and analysis processing system of a spontaneous activity monitor was used to conduct the experiment, which was conducted in a quiet environment. The mice were placed in a spontaneous activity box, and they were monitored after 1-minute adaption. A video recording apparatus was used for recording the experimental process, and the total journey, average speed and rest time of the mice in each group within 5 minutes were recorded. Between two experiments, the inner wall and bottom surface of the box were cleaned with a low-concentration ethanol solution by spraying.

(2) Elevated plus maze experiment: the specifications of the elevated plus maze include the arm length of 30 cm, the arm width of 5 cm, the closed arm height of 15 cm, and the height of the track distanced from the ground of 60 cm, and the materials for the four walls and bottom of the elevated plus maze were white poly(methyl methacrylate). One hour after the administration at 19 PM on the 8^{th} day or the 15^{th} day after the first administration, the mice were placed on the central platform with their head facing the open arms so that they were allowed to freely enter the four arms and explore for 5 min, during which their activities were recorded. The observation indicators include: the time spent in open arms of the mice and the frequency of the mice entering open arms.

(3) Pentobarbital-induced sleep experiment: the experiment was carried out in a dark environment, with keeping the room temperature at about 30° C. The mice were fasted but allowed to drink water for 12 hours before the experiment. 30 min after the administration at 9 AM on the 9^{th} day or 16^{th} day of the experiment, the mice were induced to sleep with 46 mg/kg pentobarbital sodium (by intraperitoneal injection). By timing after the injection, the time duration from the intraperitoneal injection to the disappearance of a righting reflex, i.e., Sleep Latency (SL), of each mouse was observed and recorded; after the disappearance of the righting reflex, the mice were placed on a black foam mat with their abdomen facing upward, and the time from the disappearance to the recovery of the righting reflex, i.e., Sleep Time (ST), of each mouse was observed and recorded.

### 1.6 Detection of Biochemical Indicators

On the 17^{th} day after the first administration, Blood sampling was performed from retroorbital venous plexus. 0.5 ml of the blood was taken, left to rest for 2 hours at room temperature, and centrifuged according to the conditions specified in the instructions, and the serum was separated for direct detection. Operations were made according to the instructions of the corticosterone kit (batch No: JL11918) to determine the level of the serum corticosterone.

### 1.7 Statistics of Experimental Data

The data of experimental results was expressed as Mean±Standard Error (Mean±S.E.) and statistical analysis of the data was performed by using a software. Comparisons between groups were conducted by using one-way analysis of variance (ANOVA), with P<0.05 indicating statistical significance.

### 2. Experimental Results

2.1 Effects of the Combined Use of Withania somnifera and Melatonin on the Body Weights of the Mice in Each Group

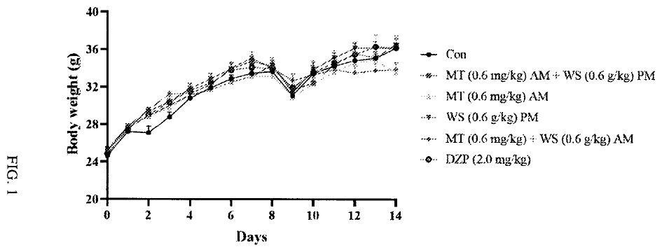

The measurement results of the body weights were shown in FIG. 1. As shown in FIG. 1, during the administration period, the body weights of the mice in each group gradually increased, and there was no significant difference in the body weights of the mice in each group. The result demonstrated that for each group, no significant toxic side effects were produced in the dose range. On the 9^{th} day, the collective decrease in body weights was due to fasting but drinking water in the evening of the previous day (on the 9^{th} day, the pentobarbital-induced sleep experiment as described in 1.5 (3) was conducted).

2.2 Effects of the Combined Use of Withania somnifera and Melatonin Combination on the Total Journey, Average Speed and Rest Time in the Spontaneous Activity Experiment for the Mice in Each Group

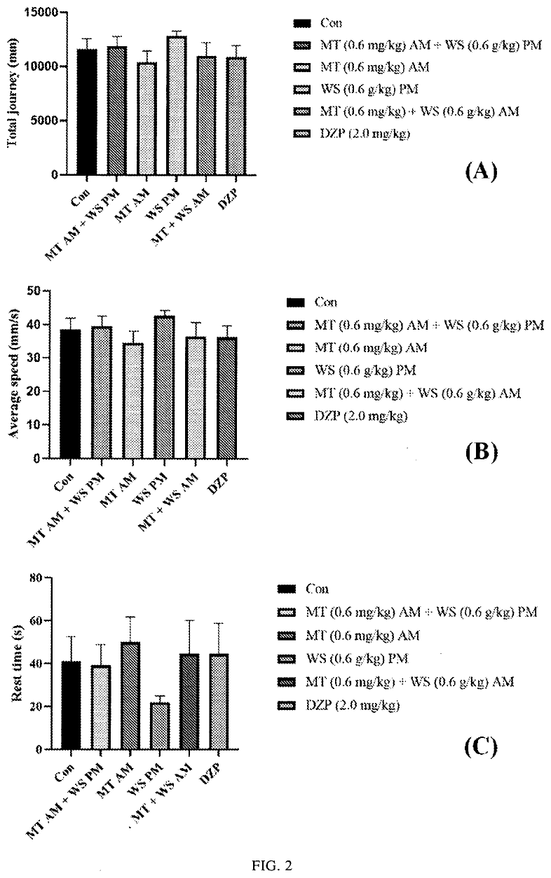

The spontaneous activity experiment may detect the spontaneous activity behaviors and exploration behaviors of the mice, and it was a method for evaluating the spontaneous behaviors, exploration behaviors and tension levels of the experimental animals in a new environment. The results of the spontaneous activity experiments were shown in Table 2 and FIG. 2.

TABLE 2
Effects of the combined use of Withania Somnifera (WS) and Melatonin
(MT) combination on the total journey, average speed and rest time
in the spontaneous activity experiment for the mice in each group

| Groups | Total journey (mm) | Average speed (mm/s) | Rest time(s) |
|---|---|---|---|
| Control | 11598.02 ± 3260.86 | 38.66 ± 10.87 | 41.07 ± 38.23 |
| MT AM + WS PM | 11885.49 ± 3089.62 | 39.62 ± 10.30 | 39.14 ± 33.71 |
| MT AM | 10385.86 ± 3481.77 | 34.62 ± 11.60 | 50.04 ± 38.82 |
| WS PM | 12819.97 ± 1432.19 | 42.73 ± 4.77 | 21.87 ± 9.32 |
| MT AM + WS AM | 10974.05 ± 3717.89 | 36.58 ± 12.39 | 44.72 ± 46.42 |
| DZP | 10897.79 ± 3471.64 | 36.33 ± 11.57 | 44.51 ± 47.44 |

As shown in Table 2 (the data on the 14^{th} day), there was no significant effect on the total journey, average speed and rest time during the spontaneous activity in all administration group as compared to the blank control group. The result demonstrated that the administration at this dose did not affect the motor behaviors of the spontaneous activity. FIG. 2 showed the effects of the combined use of Withania somnifera (WS) and Melatonin (MT) combination on the total journey (A), average speed (B) and rest time (C) in the spontaneous activity experiment for the mice in each group.

2.3 Effects of the Combined Use of Withania somnifera and Melatonin on the Time Spent in Open Arms and Frequency of Entering Open Arms in the Elevated Plus Maze Experiment for the Mice in Each Group

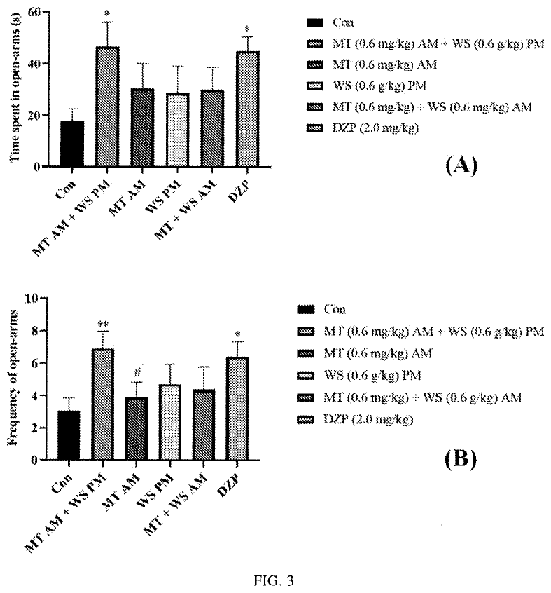

The elevated plus maze experiment was to evaluate the anxiolytic or anxiogenic effects of the test substance by utilizing a contradictory conflict state of the animals which was formed due to the animals' innate exploration behavior to a new environment and their fear to open arms suspended at a height. Normal mice exhibited a certain level of exploration activity to the open arms, while anxiety animals showed reduced exploration activity to the open arms. The results of the elevated plus maze experiment were shown in Table 3 and FIG. 3.

TABLE 3
Effects of the combined use of Withania Somnifera (WS) and Melatonin
(MT) on the time spent in open arms and frequency of entering open
arms in the elevated plus maze experiment for the mice in each group

| Groups | Time spent in open arms (s) | Frequency of entering open arms |
|---|---|---|
| Control | 18.08 ± 14.61 | 3.08 ± 2.56 |
| MT AM + WS PM | 46.62 ± 32.73* | 6.92 ± 3.69** |
| MT AM | 30.50 ± 32.06 | 3.92 ± 3.04^{#} |
| WS PM | 28.82 ± 32.85 | 4.73 ± 3.86 |
| MT AM + WS AM | 30.00 ± 26.18 | 4.40 ± 4.18 |
| DZP | 45.08 ± 18.23* | 6.42 ± 3.09* |

Note:
in comparison to the blank control group,
**represents p < 0.01,
*represents p < 0.05;
in comparison to the MT AM + WS AM group,
^{#}represents p < 0.05.

As shown in Table 3 (the data on the 15^{th} day), in comparison to the blank control group, in the “MT AM+WS PM” group and the “DZP” group, the residence time of the mice in the open arms and the frequency of the mice entering the open arms (p<0.05) were significantly increased, indicating that the “MT AM+WS PM” combined administration and the diazepam had anxiolytic effects on the mice. Other groups showed no significant difference in comparison to the blank control group.

In comparison to the “MT AM” group, the “WS PM” group and the “MT AM+WS AM” group, the “MT AM+WS PM” group showed a significant increase in the frequency of entering open arms, indicating that the anxiolytic effects of the “MT AM+WS PM” group were better than those of the “MT AM” group, “WS PM” group and “MT AM+WS AM” group. FIG. 3 showed the effects of the combined use of Withania somnifera (WS) and Melatonin (MT) on the time spent in open arms (A) and frequency of entering open arms (B) in the elevated maze experiment for the mice in each group.

2.4 Effects of the Combined Use of Withania somnifera and Melatonin Combination on the Sleep Latency and Sleep Time in the Pentobarbital-Induced Sleep Experiment for the Mice in Each Group

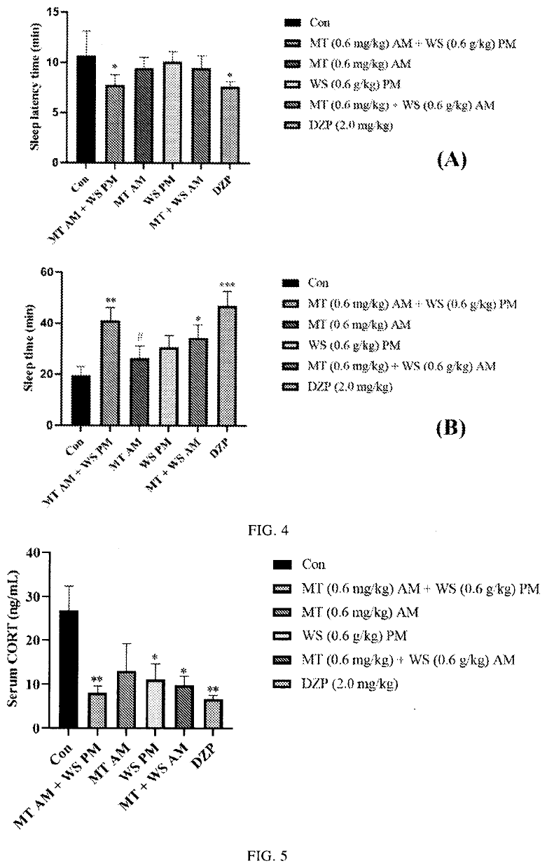

The pentobarbital-induced sleep experiment was to evaluate the effect of the test substance on improving sleep. If the test substance significantly prolonged the sleep time induced by pentobarbital or significantly shortened the sleep latency, this indicated that the test substance had the effect of improving sleep. The experimental results were shown in Table 4 and FIG. 4.

TABLE 4
Effects of the combined use of Withania Somnifera (WS) and Melatonin
(MT) on the sleep latency and sleep time in the pentobarbital-
induced sleep experiment for the mice in each group

| Groups | Sleep latency (min) | Sleep time (min) |
|---|---|---|
| Control | 10.66 ± 7.71 | 19.45 ± 11.62 |
| MT AM + WS PM | 7.80 ± 3.51* | 41.18 ± 17.60** |
| MT AM | 9.45 ± 3.62 | 26.33 ± 16.33^{#} |
| WS PM | 10.12 ± 3.09 | 30.69 ± 14.43 |
| MT AM + WS AM | 9.49 ± 3.60 | 34.38 ± 15.75* |
| DZP | 7.66 ± 1.66* | 47.03 ± 18.86*** |

Note:
in comparison to the blank control group,
***represents p < 0.001,
**represents p < 0.01,
*represents p < 0.05;
in comparison to the MT AM + WS AM group,
^{#}represents p < 0.05.

As shown in Table 4 (the data on the 16^{th} day), in comparison to the blank control group, the “MT AM+WS PM” group and the “DZP” group significantly shortened the sleep latency of the mice (p<0.05) and significantly increased the sleep time of the mice (p<0.05); although the “MT AM+WS AM” group had an effect on prolonging the sleep time (p<0.05), it had no effects on the sleep latency; the “MT AM” group and the “WS PM” group had no significant effect on both the sleep latency and the sleep time. The sleep time of the “MT AM” group, “WS PM” group and “MT AM+WS AM” group were significantly lower than that of the “MT AM+WS PM” group (p<0.05), suggesting that “MT AM+WS PM” group had the strongest effect on improving sleep, superior to that of the administrations of each of the two substances alone and the simultaneous administration of the two substances. FIG. 4 showed the effects of the combined use of Withania somnifera (WS) and Melatonin (MT) combinations on the sleep latency (A) and sleep time (B) in the pentobarbital-induced sleep experiment for the mice in each group.

2.5 Effects of the Combined Use of Withania somnifera and Melatonin on the Corticosterone in the Serum of the Mice in Each Group

Corticosterone is a corticosteroid steroid hormone with 21 carbon atoms, a glucocorticoid produced by the adrenal cortex, also called as adrenalone. Due to the increased generation of corticosteroids in response to stress, the corticosterone was used as a primary stress indicator. Moreover, in addition to the relevance to the stress, corticosterone is believed to determine the sleep and waking of humans. The experimental results were shown in FIG. 5 and Table 5.

TABLE 5
Effects of the combined use of Withania Somnifera
(WS) and Melatonin (MT) on the corticosterone
(CORT) in the serum of the mice in each group

| Groups | CORT (ng/mL) |
|---|---|
| Control | 26.89 ± 17.41 |
| MT AM + WS PM | 8.12 ± 5.34** |
| MT AM | 13.13 ± 18.40 |
| WS PM | 11.17 ± 11.26* |
| MT AM + WS AM | 9.88 ± 4.97* |
| DZP | 6.72 ± 2.87** |

Note:
in comparison to the blank control group,
**represents p < 0.01, and
*represents p < 0.05.

As shown in Table 5 (the data on the 17^{th} day), the Withania somnifera and Melatonin significantly reduced the amount of the corticosterone in the mice serum. Among them, in comparison to the “MT AM” group, the “WS PM” group and the “MT AM+WS AM” group, the “MT AM+WS PM” group had a more significant effect on the corticosterone level in the mice serum, and the results showed that the combined administration of “MT AM+WS PM” may have a stronger effect of improving sleep. FIG. 5 showed the effects of the combined use of Withania somnifera (WS) and Melatonin (MT) on the corticosterone (CORT) in the serum of the mice in each group.

To sum up, in the present application, the administration of the Withania somnifera in the evening was combined with the administration of the Melatonin in the morning, and after continuous administrations, the residence time of the mice in the open arms and the frequency of the mice entering the open arms were significantly increased, indicating that the administration way had the anxiolytic effect on the mice; further, the sleep latency of the mice was also significantly shortened, the sleep time of the mice was significantly increased, and the corticosterone content in the mouse serum was more significantly reduced, indicating that the administration way had the sleep improving effect on the mice. As to the mice, the effects of improving sleep and/or emotions achieved by the way of administrating Withania somnifera in the evening in combination with administrating Melatonin in the morning were better than the effects achieved by simultaneously administrating them in the morning and by administrating each of them alone. As to the mice, although the way of simultaneously administrating Withania Somnifera and Melatonin in the morning also exhibited the effects of improving sleep and/or improving emotion, and the effects were better than the effects achieved by administrating each of them alone, as compared to the way of administrating Withania somnifera in the evening in combination with administrating Melatonin in the morning, the effects were obviously insufficient, indicating that the way of administrating Withania somnifera in the evening in combination with administrating Melatonin in the morning could be the optimal administration way for improving sleep and/or improving emotion.

The research result of the present application provides a theoretical basis for improving sleep and/or improving emotion. Given that the murine circadian rhythm is contrary to that of humans, the research of the present application indicates that for humans, the way of administrating Withania somnifera in the morning in combination with administrating Melatonin in the evening can be the optimal administration way for improving sleep and/or improving emotion.

### Example 2: Exemplary Combined Package Product for Oral Administration

A combined package product was used for improving sleep and/or improving emotion, and the combined package product was a dietary supplement. The combined package product comprised one or more bottles of Withania somnifera dietary supplements and one or more bottles of Melatonin dietary supplements. In the instructions for using the combined package product, the combination of the administration of Withania somnifera in the morning with the administration of Melatonin in the evening was described.

### Example 3: Exemplary Combined Package Product for Oral Administration

A combined package product was used for improving sleep and/or improving emotion, and the combined package product was a dietary supplement. The combined package product was a blister package, each blister package comprising one or more capsules (or one or more tablets) of Withania somnifera dietary supplements and meanwhile one or more capsules (or one or more tablets) of Melatonin dietary supplements. In the instructions for using the combined package product, the combination of the administration of Withania somnifera in the morning with the administration of Melatonin in the evening was described.

It should be noted that in the present specification, the typical and preferred embodiments of the present application are disclosed. The above examples are only used for illustrating the technical solution of the present application but not for limiting it. While the present application is described in detail with reference to the preferred Examples, it should be understood for those skilled in the art that modifications or equivalent substitutions to the technical solution of the present application can be still made, without departing from the spirit of the application.

What is claimed is:

1. A method for improving sleep and/or improving emotions, comprising administering an effective amount of Withania somnifera and Melatonin to a subject in need thereof, wherein the Withania somnifera is administered in the morning, and the Melatonin is administered in the evening.

2. The method according to claim 1, wherein the Withania somnifera is present in the form of a medicament or a dietary supplement, and
the Melatonin is present in the form of a medicament or a dietary supplement.

3. The method according to claim 1, wherein the Withania somnifera is an extract of Withania somnifera root.

4. The method according to claim 3, wherein the extract of Withania somnifera root is administered in an amount of 100 mg to 1500 mg per day.

5. The method according to claim 4, wherein the extract of Withania somnifera root is administered in an amount of 120 mg to 1250 mg per day.

6. The method according to claim 1, wherein the Melatonin is administered in an amount of 0.3 mg to 15 mg per day.

7. The method according to claim 6, wherein the Melatonin is administered in an amount of 1 mg to 10 mg per day.

8. A combined package comprising Withania somnifera and Melatonin, wherein the Withania somnifera is present in the form of a medicament or a dietary supplement, and is administered in the morning; and
the Melatonin is present in the form of a medicament or a dietary supplement, and is administered in the evening.

9. The combined package according to claim 8, wherein the combined package is used for improving sleep and/or improving emotion of an administrated subject.

10. The combined package according to claim 8, wherein the Withania somnifera is an extract of Withania somnifera root.

11. The combined package according to claim 10, wherein the extract of Withania somnifera root is administered in an amount of 100 mg to 1500 mg per day.

12. The combined package according to claim 11, wherein the extract of Withania somnifera root is administered in an amount of 120 mg to 1250 mg per day.

13. The combined package according to claim 8, wherein the Melatonin is administered in an amount of 0.3 mg to 15 mg per day.

14. The combination package according to claim 13, wherein the Melatonin is administered in an amount of 1 mg to 10 mg per day.
